(19) United States
(12) Patent Application Publication
(10) Pub. No.: US 2018/0078749 A1
(43) Pub. Date: Mar. 22, 2018

(54) CANNULA WITH CURVED END

(71) Applicants: National Guard Health Affairs, Riyadh (SA); King Saud bin Abdulaziz University for Health Sciences, Riyadh (SA); King Abdullah International Medical Research Center, Riyadh (SA)

(72) Inventors: Muhammad Ovais ASLAM, Lahore (PK); Muhammad Arqam AWAIS, Lahore (PK); Minahil AWAIS, Lahore (PK)

(73) Assignees: National Guard Health Affairs, Riyadh (SA); King Saud bin Abdulaziz University for Health Sciences, Riyadh (SA); King Abdullah International Medical Research Center, Riyadh (SA)

(21) Appl. No.: 15/269,019

(22) Filed: Sep. 19, 2016

(51) Int. Cl. A61M 31/00

(52) U.S. Cl. CPC A61M 31/005; A61B 6/481; A61M 2210/1433; A61M 2210/1425

(57) ABSTRACT

A cannula apparatus including an outer tube with an elongated body and a groove, an inner tube disposed inside the outer tube and being manipulable through the groove, and a cap including a hole, the cap mounted at one end of the outer tube through the hole, in which the inner tube is freely slidable relative to the outer tube.

### FIELD OF THE DISCLOSURE

This disclosure relates generally to a cannula apparatus. More particularly, the present disclosure relates to a single use cannula that is configured to perform multiple procedures.

### DESCRIPTION OF THE RELATED ART

Hysterosalpingography (HSG) is a procedure that is difficult to perform and can cause laceration and trauma to the external orifice of the uterus (i.e., “external OS”) or the cervix. Also, there is leakage of contrast during procedure. There are different types of disposable cannulas available for this procedure. Most of the cannulas require the use of forceps for holding the cervix which cause pain and mild bleeding.

Several devices of various configurations have been developed to perform HSG. Many of these devices have variable stiffness and use an inflated balloon like catheters. Some of catheters are difficult to insert due to flexibility of catheter. The inflated balloon occupies the endometrial cavity and may obscure diagnostic information in this area.

A vacuum uterine cannula is difficult to use and may fail to provide a leak-proof seal. A cannula made of rigid metal can cause injury to endometrial cavity. A reusable cannula is difficult to clean for re-sterilization and can transmit diseases like AIDS and hepatitis etc.

As such, a device that performs HSG with ease and creates no trauma for the patient even in the hands of inexperienced radiologists, gynecologists, or technologists would provide many advantages. Furthermore, a cannula with which it is easy to perform HSG, and which is provided for a single use and does not require holding the cervix with forceps would provide advantages such as minimizing pain and preventing bleeding. An additional advantage would be available if the cannula can provide a tight seal with the cervix to avoid reflux of contrast.

### SUMMARY

According to an embodiment of the present disclosure, there is provided a cannula apparatus. The cannula apparatus including an outer tube with an elongated body and a groove, an inner tube disposed inside the outer tube and being manipulable through the groove, and a cap including a hole, the cap mounted at one end of the outer tube through the hole, in which the inner tube is freely slidable relative to the outer tube.

The forgoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values or dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is an exploded view of a cannula apparatus according to an exemplary embodiment of the present disclosure;

FIG. 2 is a perspective view of a cap according to an exemplary embodiment of the present disclosure;

FIG. 3 is a perspective view of the cannula in a first position according to an exemplary embodiment of the present disclosure;

FIG. 4 is a perspective view of the cannula in a second position according to an exemplary embodiment of the present disclosure; and

FIG. 5 is a perspective view of the cannula in a third position according to an exemplary embodiment of the present disclosure.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) may be practiced without those specific details. In some instances, well-known structures and components may be shown in block diagram form in order to avoid obscuring the concepts of the disclosed subject matter.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “side,” “length,” “width,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “proximate,” “minor,” and similar terms generally refer to ranges that include the identified value within a margin of 20%, 10% or preferably 5% in certain embodiments, and any values therebetween.

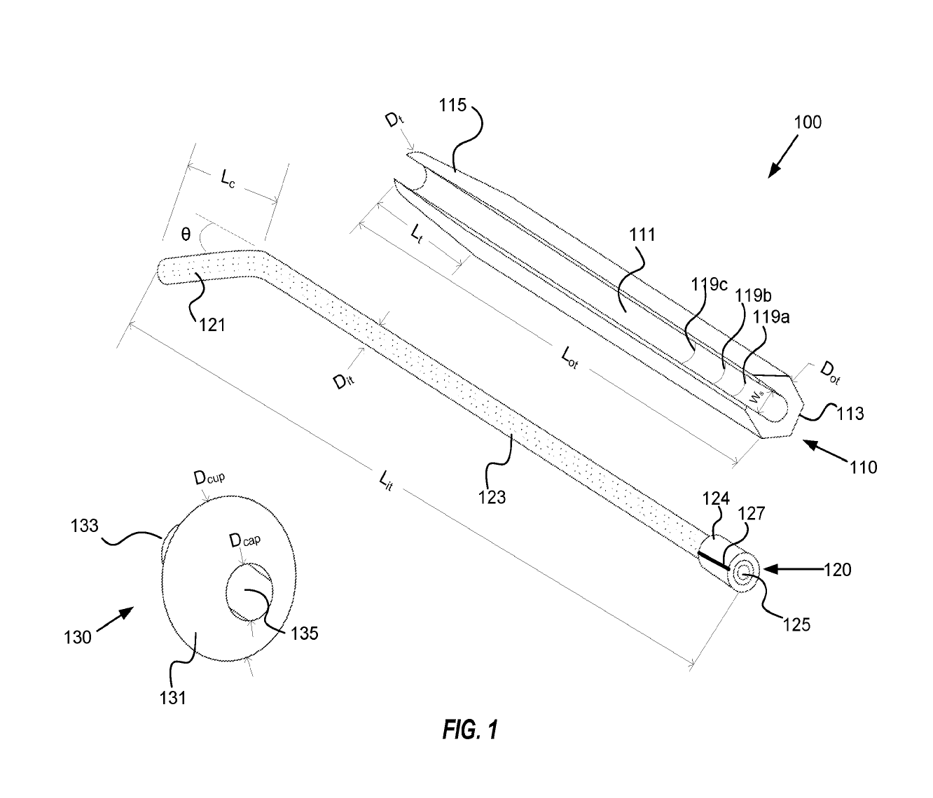

FIG. 1 is an exploded view of a cannula apparatus according to an exemplary embodiment of the present disclosure. The cannula 100 is a single use hysterosalpingography device. The cannula 100 is designed to make hysterosalpingography (HSG) simple and easy. The cannula 100 can be self-retained by a soft shield which has screw like thread allowing it to be fixed in the cervical canal. The soft shield can be made of flexible plastic or silicone.

The cannula 100 can be filled with a contrast, its tip can be inserted in the external os or cervix and the contrast can be injected in the uterine cavity of a patient. If there is a proximal tubal obstruction, a direct access to the fallopian tubes can be achieved by inserting a guide wire through the cannula 100. Thus, the cannula 100 allows passage of a guide wire assembly for interventional work, if required, without the use of forceps. The cannula 100 can be moved freely to and fro and can be rotated towards a right side or a left side (clockwise or anticlockwise) for placement of the guide wire in the isthmus of fallopian tubes.

The cannula 100 has several advantages. For example, the cannula 100 does not require holding of cervix with forceps, therefore, the HSG process can be painless with no bleeding issue. The cannula 100 can provide a tight seal with the cervix to avoid reflux of the contrast. The cannula 100 can also be used for transcervical recannulization of fallopian tubes if the fallopian tubes are blocked proximally. The cannula 100 allows a catheter and the guide wire to pass through a hollow tube of the cannula 100. The cannula 100 provides for a directional control of the guide wire as it has a bend at a distal end, which provides torque to the guide wire for entering into the ostium of the fallopian tube. Typically, the guide wire is flexible and does not have good directional control. The curved end of cannula provides direction for advancing the guide wire in the desired direction, for example, for cannulating right fallopian tube, the cure is directed to right side so the guide wire passes in the right fallopian tube easily. The cannula 100 can be rotated from outside for easy manipulation, and can be pulled out or advanced in a cavity with easy sliding motion. Thus, movements such as sliding and rotation can be performed with ease. These motions facilitate passing of guide wire for interventional procedure.

The cannula 100 includes an outer tube 110, an inner tube 120 and a cap 130. The outer tube 110 is an elongated tube with a groove 111 along the length. The outer tube 110 can be a hexagonal cross-section 113. The hexagonal cross-section 113 allows easy gripping and rotation of the outer tube 110. The outer tube 110 can be tapered at one end forming a tapered end 115. The outer surface of the tapered end 115 can be smooth in shape and the cross-section of the tapered end 115 can also be hexagonal. The groove 111 is a longitudinal cavity in the outer tube 110 with a depth extending past the center of the outer tube 110 to allow placing and guiding of the inner tube 120 inside the outer tube 110 through the groove 111. Optionally, the outer tube 110 can include one or more axial markings 119a, 119b and 119c to indicate how far the inner tube 120 tube is inserted in the external os or to measure the relative position of the inner tube 120 along the length of the outer tube 110. The axial markings 119a-119c can also serve as a measure to record the depth of the cavity of a patient. The measure can be stored and used in future by the same or different operator as a guide to operate on the patient during future visits. The axial markings 119a-119c can also serve as measures for other relevant purposes.

The outer tube 110 can be of length Lₒₜ and have an outer diameter Dₒₜ. The tapered end 115 can be of length Lₜ and have an outer diameter Dₜ. The groove 111 can have a width Wₛ, as shown in FIG. 1. According to an exemplary embodiment of the present disclosure, the outer tube 110 can be approximately 150 mm long, have an outer diameter of approximately 10 mm, the tapered end 115 can be approximately 30 mm long and the groove 111 can be approximately 5 mm wide. Furthermore, the outer tube 110 can include ridges at the proximal end to provide a click or a snap for the inner tube 120 during sliding and also to provide some resistance for the inner tube 120 so that the inner tube 120 remains inside the outer tube 110 during the procedure. It can be understood to a person skilled in art that the dimensions of the outer tube 110 can vary depending on the size of the inner tube 120 and the cap 130.

The outer tube 110 can be manufactured using standard manufacturing methods such as milling, shaping, turning, grinding, molding, casting, etc. The outer tube 110 can be made of plastic (preferably) or other metallic material such as stainless steel, copper, etc. Depending on the material chosen, appropriate manufacturing method can be used to produce the outer tube 110. For example, plastic material injection molding may be used.

The inner tube 120 is an elongated, substantially cylindrical tube. The inner tube 120 is hollow to allow the contrast to be injected in the uterine cavity of a patient and/or to allow passage of a guide wire assembly. The inner tube 120 has a curved end 121, a linear portion 123, and a hole 125 that runs throughout the length of the inner tube 120. The curved end 121 is bent at an angle θ measured with respect to the longitudinal axis of the linear portion 123 of the inner tube 120. The curved end 121 can have a rounded and smooth tip to avoid injury to cervix, cervical canal, endometrial cavity and ostium of fallopian tubes. The direction of bend of the curved end 121 can be marked on the linear portion 123 at proximal end or on a connector 124 for an operator to know the direction of the curved end 121 when inside the uterine cavity. The inner tube 120 can be rotated clockwise or anticlockwise for easy passage to guide wire tube assembly into the internal opening of fallopian tube.

The inner tube 120 can have a total length Lᵢₜ and an outer diameter Dᵢₜ. The curved end 121 can be of length L_{c}. According to an exemplary embodiment of the present disclosure, the total length Lᵢₜ of the inner tube 120 can be approximately 230 mm, the outer diameter Dᵢₜ can be approximately 4.5 mm while the inner diameter (i.e., the hole 125) can be approximately 2.5 mm. The length L_{c} of the curved end 121 can be approximately 20 mm and the angle θ can be up to 45°, preferably 25° to 30°. The bend of 25° to 30° is preferred to prevent leakage, or backflow of the contrast; as well as to avoid discomfort to the patient.

The inner tube 120 is preferably made of plastic or other soft material that can be safely inserted in a human body. However, metallic material such stainless steel can also be used. The inner tube 120 can be made of transparent material to allow visibility of the contrast passing through the hole 125. Also, if an air bubble enters in the inner tube 120, it can be seen and eliminated before the procedure to avoid false results as the air bubbles can look like lesion. The inner tube 120 is stiff enough that it cannot bend while operating on a patient. However, the bend of the curved end 121 can be changed manually on application of reasonable force to change the angle of the curved end 121.

At the proximal end (opposite of the curved end 121) of the inner tube 120, a connector 124 can be attached. The connector 124 is an attachment such as a luer-lock that allows leak-free connection with a syringe. Furthermore, the connector 124 includes a direction mark 127 to show the direction of bend of the curved end 121. The direction mark 127 can act as an indicator, especially, when the curved end 121 is inserted in the cervix and the curved end 121 is not visible from outside. According to an exemplary embodiment, the connector 124 can be approximately 17 mm long with an outer diameter of approximately 6 mm and an inner diameter of approximately 4.5 mm. The proximal end of the connector 124 can include a collar of size approximately 8 mm (as may be present with the syringe) for leak-free connection with the syringe.

The cap 130 is an attachment to retain the inner tube 120 inside the outer tube 110. The cap 130 can also provide a water tight seal at the cervix. Also, if required, a slight pull to the cervix can be achieved to straighten the utero cervical junction for better image quality and for further passage of the inner tube 120, for example, inside the endometrial cavity for canalization of ostium of fallopian tube. The cap 130 can be made of soft PVC plastic or soft medical grade silicon. The cap 130 can be made of transparent plastic material to see spillage/reflux of contrast out from the uterine cavity.

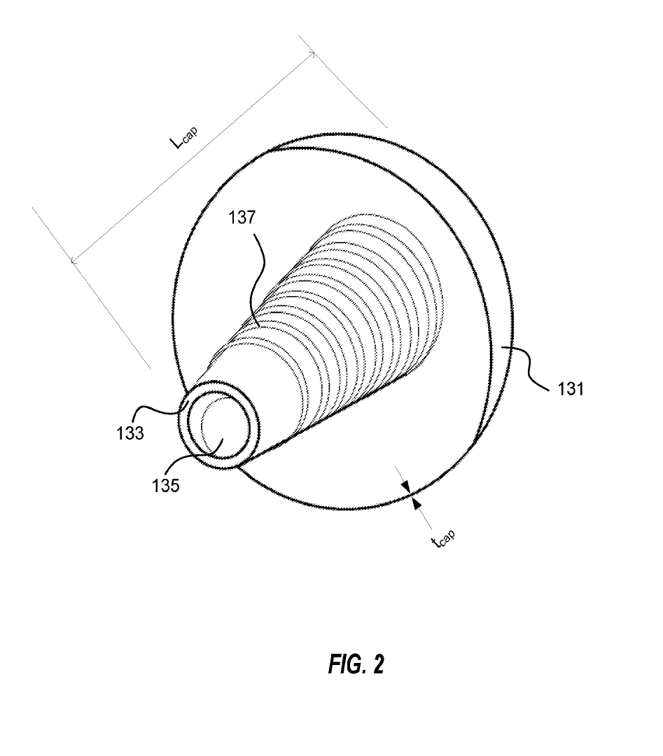

As shown in FIGS. 1-2, the cap 130 has a cone 133 and a cup 131 attached at the base of the cone 133. The cap 130 has a cavity extending therethrough and has a tapered hole 135 with diameter D_{cap} at the base of the cone 133. The cup 131 has a curved shape being convex on the proximal end and being concave on the distal end and can be of diameter D_{cup} at its largest. The cup 131 can capture spillage/reflux of contrast out from the uterine cavity. Referring to FIG. 2, the outer surface of the cone 133 includes threads 137. The cap 130 can be of length L_{cap}. The length L_{cap} can be substantially similar to the length Lₜ of the tapered end 115 of the outer tube 110. When assembled, the cap 130 can tightly fit on the tapered end 115 of the outer tube 110 and the apex of the cone 133 can fit tightly on the inner tube 120, while pointing towards the curved end 121 of the inner tube 120. When disassembling the cannula 100, the cap 130 can be detached by sliding the cap 130 out of the outer tube 110.

According an exemplary embodiment, the dimensions of the cap 130 can be as follows. The diameter of the cone 133 at the apex can be approximately 4 mm gradually increasing to approximately 5 mm at the base. The length L_{cap} of the cap 130 can be approximately 30 mm. The diameter D_{cap} of the tapered hole 135 can be approximately 8 mm at its largest gradually tapering towards a distal end. The threads 137 can be formed along the cone 133 for a length of up to 23 mm. The diameter D_{cup} of the cup 131 can be approximately 27 mm and the thickness t_{cup} (shown in FIG. 2) of the cup 131 can be approximately 3 mm. It can be understood by a person skilled in art that the dimensions of the cap 130 can be easily modified according to the size of the outer tube 110 and/or the inner tube 120.

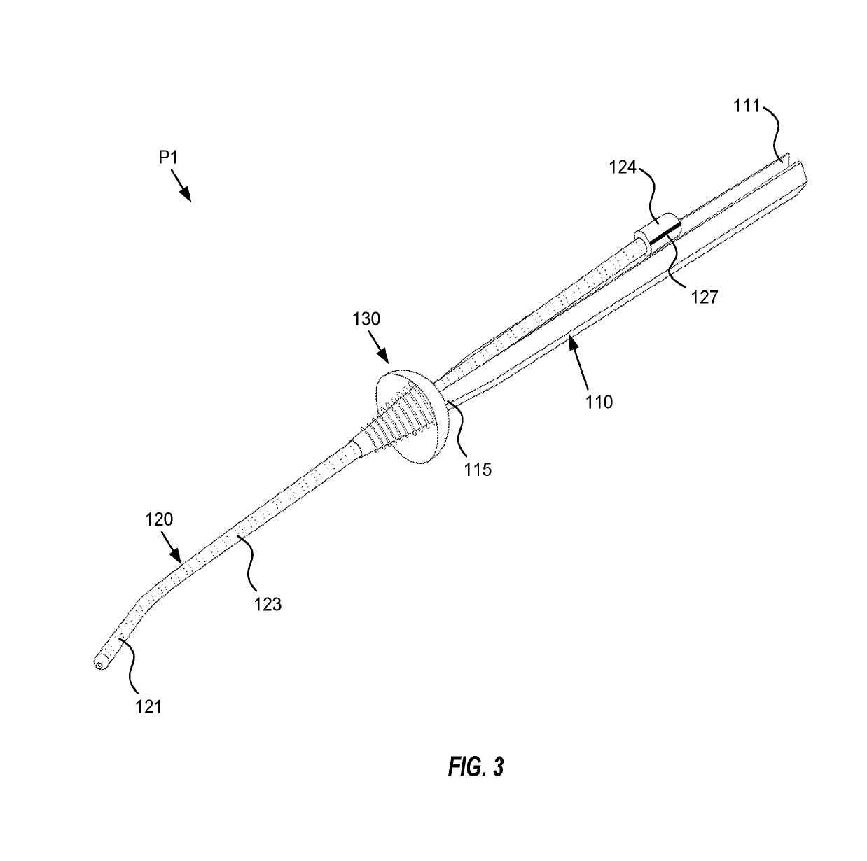

FIG. 3 is a perspective view of the cannula in a first position P1 according to an exemplary embodiment of the present disclosure. The first position P1 represents a first step of operating the cannula 100. In the first position P1, the curved end 121 of the inner tube 120 is inserted in the external os or cervix by pushing the inner tube 120 forward by way of the groove 111 of the outer tube 110 and the cap 130 occupies a position approximately at the midpoint of the inner tube 120. If the cap 130 is close to the curved end 121, the view of the external os will be blocked and affect the visibility while entering the inner tube 120 into the external os or cervix. Thus, because the curved end 121 is inserted into the external os while the cap 130 does not block the external os, the first position P1 gives good visibility of the cervical opening with no visual obstruction from the cap 130.

Further, in the first position P1, the proximal end of the inner tube 120, where the connector 124 is attached, is slightly lifted out of the outer tube 110 through the groove 111 and the connector 124 projects out from the groove 111 due to the size of the connector 124. Also, the connector 124 projecting out from the groove 111 provides an easy access, gripping and movement of the inner tube 120. Thus, the cannula 100, in the first position P1, can be used for initial examination, for example, to measure the length and direction of the cervical canal and uterus without the use of an uterine sound (a sound based test to determine the uterus shape and size) and ascertains the position of uterus by forwarding the inner tube 120 into uterine cervix through the ostium.

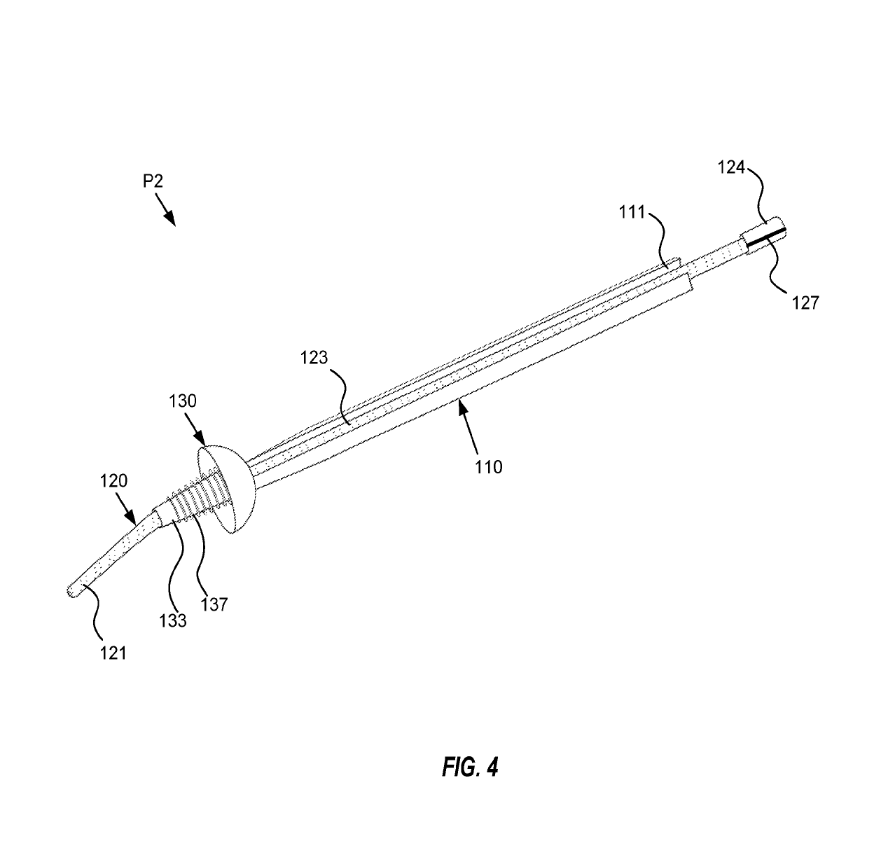

FIG. 4 is a perspective view of the cannula in a second position P2 according to an exemplary embodiment of the present disclosure. In the second position P2, the configuration of the cannula 100 is such that the apex of the cone 133 of the cap 130 is positioned close to the curved end 121 of the inner tube 120, the linear portion 123 rests inside the outer tube 110 without protruding through the groove 111, and the connector 124 projects out from the proximal end of the outer tube 110. The cannula 100 can occupy the second position P2 (from the first position P1) by holding the connector 124 in place and pushing the outer tube 110 towards the curved end 120 of the inner tube 120 till the apex of the cone 133 of the cap 120 is close to the curved end 121.

When operating on the patient, the cannula 100 is pushed until the cap 130 reaches the external os and when the cap 130 reaches the external os, the cap 130 can be passed into cervical canal by clockwise rotation. The clockwise rotation of the cap 130 can be achieved by rotating the outer tube 110. For example, for nulliparous procedure, the cap 130 can be rotated for three complete rotations. Similarly, for dilated OS and nine rotations for incompetent OS, the cap 130 can be rotated for six and nine complete rotations, respectively.

Once the cap 130 is inserted into the cervical canal, the contrast can be injected and films can be taken. The cup 131 of the cap 130 can fitted against the vagina to capture any spill or leakage of the contrast in the cup 131. The second position P2 can be particularly useful to perform diagnostic HSG. After completion of the HSG procedure (or other procedures), the cap 130 can be removed from the cervical canal by anticlockwise rotation of the outer tube 110.

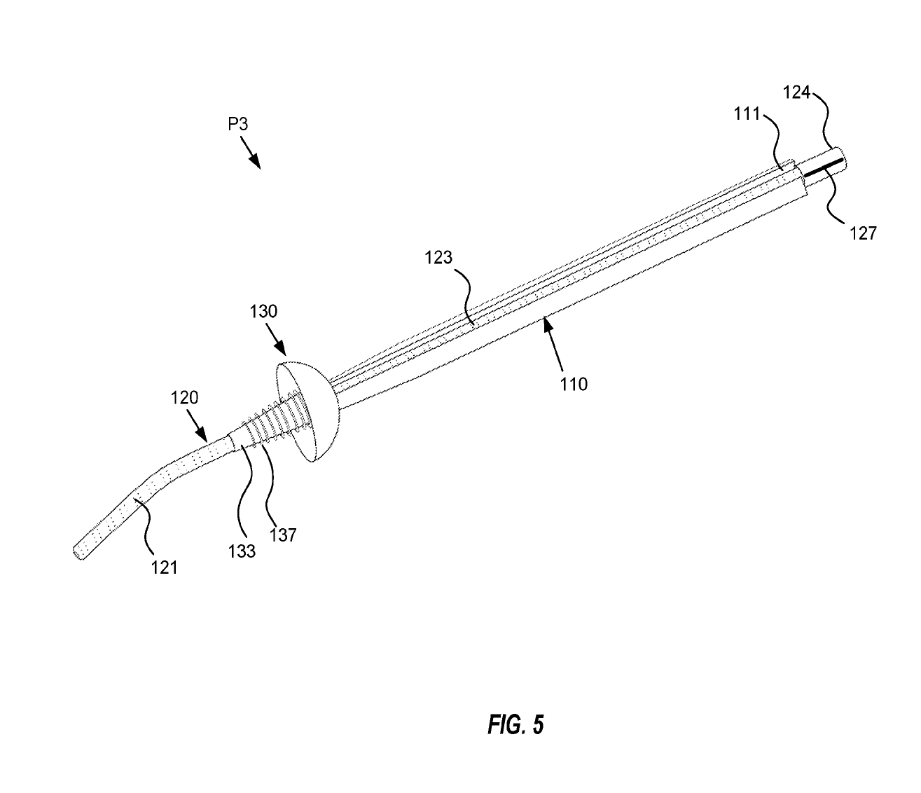

FIG. 5 is a perspective view of the cannula in a third position P3 according to an exemplary embodiment of the present disclosure. In the third position P3, the configuration of the cannula is such that the apex of the cone 133 of the cap 130 is slightly retracted from the curved end 121, and the connector 124 at the proximal end of the inner tube 120 is abutted to the proximal end of the outer tube 110. The third position P3 can be reached after completion of the HSG and/or when a transcervical recannulization of fallopian tubes needs to be performed.

There are several advantages of using the cannula 100. By using cannula 100, procedures such as HSG can be performed without the need of a tenaculum or other cervix holding forceps. The inner tube 120 allows the contrast to be injected in the uterine cavity of a patient and/or allows for passage of a guide wire assembly. The inner tube 120 has a curved end 121 to prevent leakage, or backflow of the contrast as well as to avoid discomfort to the patient. The curved end 121 has a rounded and smooth tip to avoid injury to cervix, cervical canal, endometrial cavity and ostium of fallopian tubes. The direction of bend of the curved end 121 is marked on the connector 124 at proximal end for an operator to know the direction of the curved end 121 when inside the uterine cavity. The connector 121 can also act as a luer-lock to connect with a syringe.

The outer tube 120 has the hexagonal cross-section 113 that allow easy gripping and rotation of the outer tube 110. Furthermore, the outer tube 110 includes the axial markings 119a, 119b and 119c to indicate how far the inner tube 120 tube is inserted in the external os or to measure the relative position of the inner tube 120 along the length of the outer tube 110.

The cap 130 retains the inner tube 120 inside the outer tube 110. The cap 130 includes threads that provide a water tight seal at the cervix and also facilitate a slight pull to the cervix to straighten the utero cervical junction. The cup 131 captures spillage/reflux of contrast out from the uterine cavity.

In addition, the inner tube 120 and the cap 130 can be made of transparent material to see the contrast or guidewire being passed through the inner tube or to see if there is any air bubble or leakage of the contract in the cup 131.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures. For example, this technology may be structured for cloud computing whereby a single function is shared and processed in collaboration among a plurality of apparatuses via a network.

What is claimed is:

1. A cannula apparatus comprising:
an outer tube with an elongated body and a groove;
an inner tube disposed inside the outer tube and being manipulable through the groove; and
a cap including a hole, the cap mounted at one end of the outer tube through the hole,
wherein the inner tube is freely slidable relative to the outer tube through the hole.

2. The apparatus according to claim 1, wherein the cap comprises a cone with a cup integrally attached to a base of the cone.

3. The apparatus according to claim 2, wherein the cup has a curved shape with the base of the cone attached in a valley of the cup.

4. The apparatus according to claim 3, wherein the hole of the cap is a tapered hole extending from the cup to an apex of the cone such that the tapered hole gradually decreases in diameter from the cup to the apex of the cone.

5. The apparatus according to claim 2, wherein the cone has external threads to provide a tight seal.

6. The apparatus according to claim 2, wherein the cup has a curved shape and a concave side of the cup faces away from a proximal end of the outer tube and creates a seal to avoid spillage of a contrast during operation.

7. The apparatus according to claim 1, wherein the cap holds the outer tube and the inner tube together during movement of the inner tube with respect to the outer tube.

8. A cannula apparatus comprising:
an outer tube including a groove along a length of the outer tube; and
an inner tube including a curved end and a linear portion, the linear portion being slidably disposed inside the outer tube and being controllable through the groove.

9. The apparatus according to claim 8, wherein the outer tube has a hexagonal cross-sectional shape.

10. The apparatus according to claim 9, wherein the outer tube includes one or more axial marking to indicate a position of the inner tube with respect to a longitudinal axis of the outer tube.

11. The apparatus according to claim 8, wherein the curved end of the inner tube is bent at an angle of 25 to 30° with respect to a longitudinal axis of the linear portion.

12. The apparatus according to claim 8, the linear portion of the inner tube slides linearly inside the outer tube.

13. The apparatus according to claim 8, further comprising a connector attached to a proximal end of the inner tube.

14. The apparatus according to claim 13, wherein the connector includes a direction mark to indicate a direction of the curved end of the inner tube.

15. The apparatus according to claim 14, further comprising a cap mounted on the outer tube and the inner tube such that the inner tube slides freely.

16. The apparatus according to claim 15, wherein the cap holds the outer tube and the inner tube together during movement of the inner tube with respect to the outer tube.

17. The apparatus according to claim 16, wherein the cannula is configured to occupy a plurality of positions including a first position, a second position and a third position.

18. The apparatus according to claim 17, wherein, in the first position, a midpoint of the inner tube is at the cap, and the proximal end of the inner tube with a connector projects outside the groove.

19. The apparatus according to claim 17, wherein, in the second position, the curved end of the inner tube is closest to the cap, and the connector does not touch the outer tube.

20. The apparatus according to claim 17, wherein, in the third position, the connector is closest to the proximal end of the outer tube and the cap is spaced apart from the curved end of the inner tube.
